(19) United States
(12) Patent Application Publication
(10) Pub. No.: US 2008/0255409 A1
(43) Pub. Date: Oct. 16, 2008

(54) PROCESS TO IMPROVE THE PROFITABILITY OF TESTS OR TREATMENTS WITH ENDOSCOPY CAPSULES

(76) Inventors: Rainer Graumann, Hochstadt (DE); Rainer Kuth, Hochstadt (DE)

Correspondence Address: SCHIFF HARDIN, LLP;PATENT DEPARTMENT, 6600 SEARS TOWER, CHICAGO, IL, 60606-6473, US

(21) Appl. No.: 12/101,244

(22) Filed: Apr. 11, 2008

(30) Foreign Application Priority Data
Apr. 12, 2007 (DE) 10 2007 017 267.4

(51) Int. Cl. A61B 1/00

(52) U.S. Cl. 600/101

(57) ABSTRACT

In a method for improving profitability of a medical procedure supervised through a medical facility, the medical procedure including ingestion of an endoscopy capsule by a patient of the medical facility, the endoscopy capsule is retrieved after being naturally voided by the patient after the endoscopy capsule traverses the gastrointestinal tract of the patient, and a reward is paid to a person who returns the retrieved endoscopy capsule to a location associated with the medical facility. An endoscopy capsule usable with this procedure has a capsule body containing medical data-gathering components, and an exchangeable cover that covers the capsule body that is formed of a material allowing removal of the cover after the capsule has passed through the gastrointestinal tract of the patient, so that a new cover can be provided on the capsule body.

### BACKGROUND OF THE INVENTION

1. Field of the Invention

The present invention concerns a process to improve the profitability of tests, specifically screening tests, or treatments, of the type wherein an endoscopy capsule ingested by the patient traverses the gastrointestinal tract of the patient and is voided in a natural manner. In addition, the invention concerns an endoscopy capsule for use within the gastrointestinal tract of a patient as well as a process to reuse endoscopy capsules for use in the gastrointestinal tract of a patient.

2. Description of the Prior Art

Endoscopy capsules are known for testing and for therapy of the gastrointestinal tract, where, for example, a video unit may capture views of the interior of the body. The patient swallows the capsules, and they traverse the gastrointestinal tract. After eight hours, for example, the endoscopy capsule is voided naturally via the anus.

The endoscopy capsules known today are expensive (roughly EUR 500 each) and normally may be used only once.

This is in contrast to the desire to open the field for so-called mass screening technologies by the use of such endoscopy capsules. This also competes with the requirement that such a screening must be low cost due to the normally low rate of positive findings. This generates the desire for technology to permit a reuse of endoscopy capsules. To this end, it has been proposed to use a toilet, designed so that the capsules in the stool could be noted, captured and cleaned. However, this requires the patient to use a specially designed and expensive toilet, with additional problems generated by variations in dwell time of the capsule within the patient.

### SUMMARY OF THE INVENTION

An object of the present invention is to provide an option to facilitate the reuse of endoscopy capsules that is simple to use.

This is achieved in accordance with the invention by a method wherein the profitability of tests and treatments making use of procedures of the type described above is improved by offering a reward to the person submitting an endoscopy capsule for reuse after voiding.

Such an advantageous procedure gives the patient or other persons taking possession of a used endoscopy capsule an incentive to ensure that the relatively expensive endoscopy capsule is reused. This process permits the multiple reuse of an appropriately designed endoscopy capsule, such that the costs for a single application are reduced. It is also advantageous that this reduces the amount of waste requiring special treatment that normally accrues from such technologies. The incentive proposed by the invention could also facilitate the use of endoscopy capsules in mass screening by way of active participation of the general population.

In an embodiment of the invention the patient obtains a reward when he or she returns the capsule to a reuse center. Thus, the patient is provided with the address of a reuse center when he or she swallows the capsule, to which the patient may then return the endoscopy capsule after it has been voided after use. The appropriate cleaning may then take place at that location. If the patient has mailed the endoscopy capsule to the reuse center, he or she will be paid the specified reward.

It is particularly advantageous to provide the patient with a mailer and/or a form for the return of the capsule at the time when the endoscopy capsule is provided. The mailer may have a preprinted return address for the reuse center, for example. The form may provide space for the patient to provide data regarding the patient, the circumstances and the reward. Such an arrangement will make it easier for the patient to return the capsule, since it reduces the effort significantly.

The packaging used here may also include a mechanism to retrieve the capsule. Such a mechanism may include a magnet, for example. Such a mechanism enables the patient to search the voided stool for the endoscopy capsule, with better sanitation. The mechanism may be designed such that the capsule is retrieved directly into the packaging, such that it merely needs to be closed and mailed. This comfortable method enables the patient to send the endoscopy capsule back for reuse and to obtain the reward.

The reward may be in cash and/or virtual cash, specifically in the form of a coupon and/or an option for stock and/or frequent flier miles, and/or an object, such as a product from a catalog. The monetary amount should be set such that the reuse system is fiscally sound and that the patient still has sufficient incentive to send the capsule in for reuse. The rewards could also be based on exchange programs with firms that offer goods or bonus programs demanded by the patients. For example, it could be based on cooperation with an airline, such that a patient returning a capsule is credited with a certain number of frequent flyer miles.

In another embodiment of the invention, a previous deposit is returned as part of the reward. This method would integrate a deposit system into the process of the invention. A deposit is an additional incentive to return the capsule for reuse. Deposit systems are widely proven and used in other areas.

Even with the above procedure, it is possible that the patient may not send or be able to send the endoscopy capsule, but a third person may come into possession of a used endoscopy capsule. In that case, provision may be made to reward the finder of the endoscopy capsule for the return for reuse with the same or a different reward. For example, it is possible that used endoscopy capsules are not retrieved and reach a sewage treatment plant. The endoscopy capsules may be found there in filters or the like. The operator of the sewage treatment plant may then forward the accumulated endoscopy capsules for reuse by sending them back, for example. In this case, the same reward as for a patient may apply; alternatively, a different reward may be offered.

The invention also indicates that the reward may be reduced, if the capsule cannot be reused. This would apply in particular to an instance where the patient returning the capsule subjects it to inappropriate treatment. This approach could reduce excessive outlays for defective capsules.

In addition, the invention concerns an endoscopy capsule for use in the gastrointestinal tract of a patient that is characterized by being enclosed in an exchangeable and at least partly transparent closed cover of the endoscopy capsule, where the cover is resistant to the environment within the gastrointestinal tract.

A further noteworthy circumstance for the reuse of endoscopy capsules relates to the appropriate cleaning and sterilization of the capsule prior to use by another patient. Given the complex and partly delicate surface of the endoscopy capsules, complicated cleaning processes are known, which are expensive and technically difficult to implement. The endoscopy capsule of the present invention relates to this. It proposes to use a single-use cover, which may be removed easily in a reuse center, to be replaced by another cover. A cheap, replaceable component of the endoscopy capsule protects the expensive components from contamination and facilitates inexpensive reuse. Moreover, the exchange is technically easy to install, given that it merely involves the removal of a cover and the placement of another cover.

The endoscopy capsule is covered completely by the cover, which is also resistant to the conditions in the interior of the gastrointestinal tract. The interior of the cover is thus protected from dirt and damaging influences, such that other damages to the endoscopy capsule are prevented. Moreover, the cover is at least partly transparent, such that a testing tool, such as a video camera, or a treatment tool, such as a laser or the like, is not hindered in its operation. The cover may also be completely transparent. For example, if there is a video camera at each end of the elongated endoscopy capsule, the cover may be transparent at these ends, but not transparent in the middle. In any case, care must be taken that highly different refraction indices do not lead to distortions of the photos taken.

The material of the cover needs to be biocompatible to preclude discomfort to the patient. Likewise, the electrical properties of the capsule should not be affected.

In a further embodiment of the endoscopy capsule of the invention, a cover is provided in the form of a protective lacquer and/or a foil. The protective lacquer may be applied by known application methods (spray, pour, submersion, etc.). The foil may use a plastic foil consisting of long-chain plastics, which are also applied with essentially known processes (lamination, shrink, etc.). Of course, it is also possible to use other suitable materials for the cover.

Furthermore, the design of the cover should be chosen such that the removal or decomposition of a used cover can be facilitated in a particularly simple manner. A particularly simple case is to rip the foil open and to pull the foil off. The invention also proposes that the cover may loosen or dissolve, when the cover heats to temperatures that exceed body temperature. Such a design may use, for example, a thermoplastic foil or a protective lacquer with a low dissolution or melting temperature. Once a returned endoscopy capsule is received in the reuse center, the capsule need only be heated to remove the old cover. The new cover may then be applied. Of course, the treatment temperature in question should exceed the temperature in the body's interior, so that the dissolution or removal process does not start within the body.

A different embodiment may also provide for a cover that can be removed by a chemical reaction with a removal material. The removal material may be any chemical compound which dissolves the cover, but does not attack the remaining endoscopy capsule. This may conceivably consist of known lacquer removers or solvents, whether liquid or gaseous. Likewise, removal by reactive plasma is also conceivable. This method may also remove the cover for reuse without problems.

The cover should have a thickness that precludes any hygienic concerns, which implies that no body fluids reach the covered part of the endoscopy capsule. This means that the cover may have a homogeneous thickness of ≧0.1 mm.

In addition to the endoscopy capsule, the invention also concerns a process for the reuse of endoscopy capsules for use in the gastrointestinal tract of a patient, which is characterized by removing an at least partly transparent cover, which had been affixed prior to ingestion of the capsule by the patient, which can be exchanged, and which is resistant to the influences in the interior of the gastrointestinal tract, after use of an endoscopy capsule in the gastrointestinal tract and replacing it with a new cover. This process may use, for example, the endoscopy capsule of the invention as described in the previous paragraphs.

In order to facilitate the cheapest and technically simplest embodiment possible for the replacement of the covers, several alternatives may be considered.

For example, in order to remove the temperature-sensitive cover, the system may be designed to heat the capsule to a certain temperature above body temperature in order to loosen or dissolve the cover.

As an alternative, in order to remove the cover, the capsule may be subjected to a removal compound to remove the cover by a chemical reaction.

A third alternative for the process would be to open a cover consisting of a foil by opening the foil and removing it.

The application of a new foil may be handled in several processes that are essentially known. For example, the cover may be sprayed on, laminated on and/or layered on. The respective processes are essentially known and need not be discussed in detail here.

### BRIEF DESCRIPTION OF THE DRAWINGS

FIG. 1 schematically illustrates an embodiment of a method to improve profitability with regard to the use of an endoscopy.

FIG. 2 is a section through an endoscopy capsule according to the invention.

FIG. 3 illustrates a first embodiment of the process to reuse the endoscopy capsules for use in the gastrointestinal tract of a patient.

FIG. 4 illustrates a second embodiment of the process to reuse the endoscopy capsules for use in the gastrointestinal tract of a patient.

### DESCRIPTION OF THE PREFERRED EMBODIMENTS

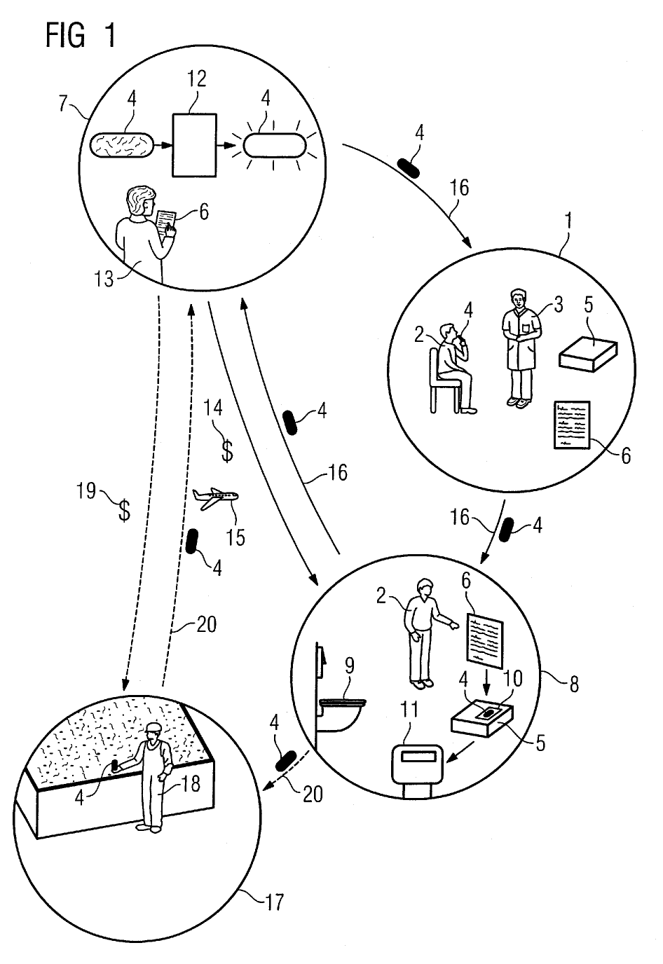

FIG. 1 schematically illustrates an embodiment of the invention designed to improve the profitability of tests, specifically screening tests, or treatments, where an endoscopy capsule ingested by the patient traverses the gastrointestinal tract of the patient and is voided by natural means. It depicts a physician's office 1, in which a patient 2 ingests an endoscopy capsule 4 under the supervision of physician 3. The endoscopy capsule 4 traverses the gastrointestinal tract of patient 2 and registers photos by means of a video camera, for example, which are transmitted by wireless transmission to a receiver (not depicted here) worn by patient 2 to be stored there, or dispenses a treatment. In this process, physician 3 provides patient 2 with packaging 5 and form 6, which are pre-addressed to a reuse center 7 so as to facilitate the return of endoscopy capsule 4 by patient 2. After the test/treatment, the patient returns to his or her living quarters 8, where the patient voids the endoscopy capsule 4 in a toilet 9 after some time, such as eight hours. Packaging 5 includes device 10 to retrieve the endoscopy capsule 4. This may be a collection magnet, for example. This enables patient 2 to remove the endoscopy capsule 4 from the stool, and the endoscopy capsule 4 is transferred directly into odor-proof packaging 5. In addition, patient 2 fills out a form 6, which the patient also inserts into an appropriate slot of packaging 5. The patient enters personal data, data regarding the desired reward and/or the treatment on form 6. Patient 2 then drops packaging 5, which is addressed to reuse center 7, into mail box 11, such that endoscopy capsule 4 will be forwarded to reuse center 7. There, capsule 4 is cleaned or again sterilized by suitable equipment 12 or specialists for reuse. Staffer 13 processes form 6 and transfers the appropriate reward to patient 2, here in the form of a monetary payment 14 or frequent flyer miles 15. Thus, patient 2 receives a reward for returning voided endoscopy capsule 4 for reuse.

The appropriately cleaned capsule 4 may then be reused and is again returned to physician's office 1. This creates the return flow shown by arrows 16.

In cases where patient 2 does not wish to return endoscopy capsule 4 or cannot find it in the stool, the endoscopy capsule 4 may be transported to another place, such as waste treatment plant 17, where a finder 18 retrieves it from a filter, for example. It is particularly advantageous to inform operators and staff of a waste treatment plant regarding the reuse program defined by the invention, because lost endoscopy capsules 4 are often found in waste treatment plants. Thus, finder 18 is informed and returns endoscopy capsule 4 to reuse center 7. He also obtains a reward for the return, here monetary payment 19. This is another pathway by which endoscopy capsule 4 can be returned for reuse, arrows 20.

It is also possible in the framework of the process of the invention that a portion of the reward obtained by patient 2 is the return of a deposit that the patient made in the physician's office 1 prior to treatment. This method provides an additional incentive.

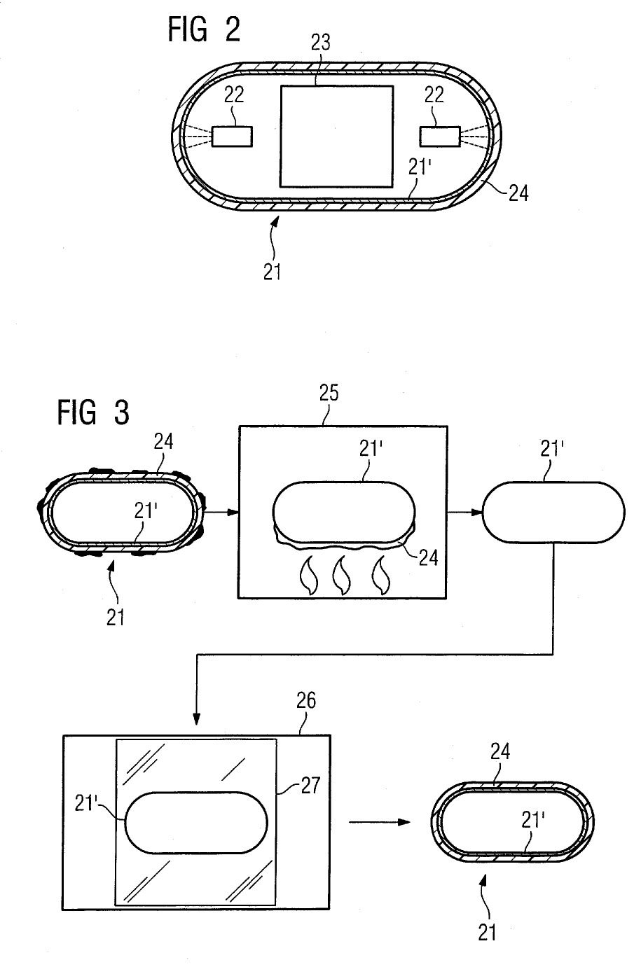

FIG. 2 shows endoscopy capsule 21 of the invention in a cross section. In this case, it refers to a testing capsule for gastroscopy. To this end, endoscopy capsule 21 is equipped with a video camera 22 at its front and reverse end, which may take pictures in the stomach of a patient, for example, and transmit them to the physician by appropriate electronics 23. Of course, endoscopy capsule 21 may also contain various other elements, which are well known and need not be discussed here in detail. The endoscopy capsule 21 is completely covered by cover 24, which is transparent, at least in the area of the field of vision of video cameras 22, where care needs to be taken to avoid distortions caused by different indices of refraction between the transparent segments of the capsule housing and the transparent cover 24. Cover 24 is also biocompatible and resistant to the environmental impacts found in the gastrointestinal tract. Cover 24 thus protects capsule 21′ from damage and contamination. In the present example, cover 24 is 0.3 mm thick and consists of a thermoplastic plastic foil made of long-chained polymers. However, it is also conceivable to use a protective lacquer.

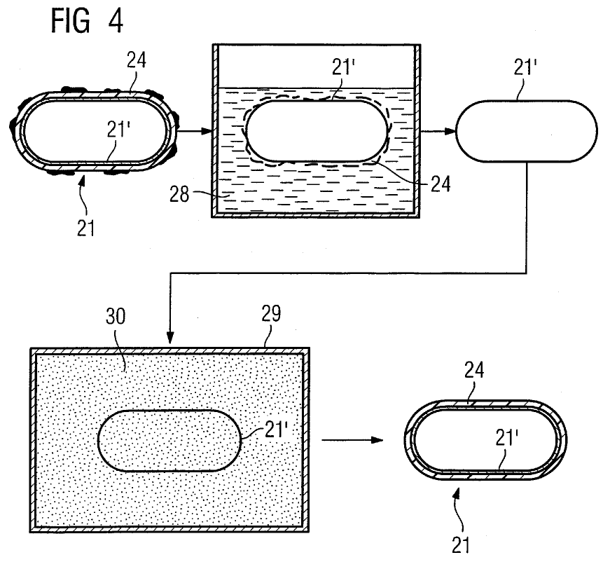

Cover 24 can be exchanged, which means that a used cover 24 may be removed and replaced by a new cover 24. This may be done in the reuse center 7, for example. Appropriate processes for reuse of endoscopy capsule 21′ are shown in FIGS. 3 and 4.

Once endoscopy capsule 24 has traversed the gastrointestinal tract of a patient, it is contaminated hygienically and cannot be used directly for another patient. However, since the cover 24 has protected the interior of endoscopy capsule 24 from contamination and damage, endoscopy capsule 21 can be used again after cover 24 has been replaced. FIG. 3 shows a process by which the cover 24 is removed by heat impacts. Thus, the contaminated endoscopy capsule 21, possibly after basic cleaning, is inserted into an oven 25, where it is heated to a certain temperature, at which the cover 24 loosens or dissolves. Once the cover 24 has been removed totally, the remainder of the endoscopy capsule 21′ is inserted into a covering unit, such as a lamination unit 26, where it is covered by a new thermoplastic foil 27, which then forms a new cover 24.

FIG. 4 shows a process in which the cover 24 is removed by a chemical removal compound. Contaminated endoscopy capsule 21 is immersed in removal compound 28, which reacts with the material of the cover, but not with the remaining components of endoscopy capsule 21. Once the cover 24 is removed completely, the uncovered endoscopy capsule 21′ is immersed in covering unit 29, where it is covered uniformly with material 30 to form the cover, such as a lacquer, such that a new cover 24 has formed on capsule 21.

Of course, it is also possible to tear a foil-type cover 24 and to remove it by hand.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for improving profitability of a medical procedure supervised through a medical facility, the medical procedure including ingestion of an endoscopy capsule by a patient of the medical facility, said method comprising the steps of:
retrieving the endoscopy capsule after natural voiding of the endoscopy capsule by the patient after the endoscopy capsule traverses the gastrointestinal tract of the patient; and
paying a reward to a person who returns the retrieved endoscopy capsule to a location associated with the medical facility.

2. A method as claimed in claim 1 comprising associating an endoscopy capsule reuse center with the medical facility, and paying said reward to said person upon return of the retrieved endoscopy capsule by said person to said endoscopy capsule reuse center.

3. A method as claimed in claim 2 comprising, at a time associated with ingestion of the endoscopy capsule by the patient, providing the patient with at least one of return packaging and a return form for returning the retrieved endoscopy capsule to the endoscopy capsule reuse center.

4. A method as claimed in claim 3 comprising including a retrieval device in said return packaging to facilitate retrieval of the endoscopy capsule.

5. A method as claimed in claim 4 comprising providing a magnet as said retrieval device

6. A method as claimed in claim 1 comprising paying said reward as a remuneration selected from the group consisting of a monetary payment, a stock option, frequent flyer miles, and a product from a catalog.

7. A method as claimed in claim 1 comprising requiring payment of a deposit at a time associated with ingestion of the endoscopy capsule by the patient, and paying said reward as at least a partial return of said deposit.

8. A method as claimed in claim 1 comprising setting an amount of said reward dependent on whether said person returning the retrieved endoscopy capsule is the patient or a person other than the patient.

9. A method as claimed in claim 1 comprising setting an amount of said reward dependent on whether the retrieved and returned endoscopy capsule is in a condition permitting reuse of the retrieved and returned endoscopy capsule.

10. An ingestible endoscopy capsule comprising:
a capsule body having a size and a shape configured for ingestion by a patient and natural passage of the capsule body through the gastrointestinal tract of the patient, said capsule body containing at least one data acquisition component that acquires medically relevant information from the patient during said passage of the capsule body through the gastrointestinal tract of the patient; and
an exchangeable cover covering said capsule body, said cover being comprised of a material that allows acquisition of said medically relevant information by said component in said capsule body through said cover, and that is resistant to the environment within said gastrointestinal tract of the patient, said cover being configured for removal from said capsule body after said passage of said capsule body through the gastrointestinal tract of the patient.

11. An endoscopy capsule as claimed in claim 10 wherein said cover comprises a protective layer on an exterior of the capsule body, selected from the group consisting of a lacquer layer and a foil layer.

12. An endoscopy capsule as claimed in claim 11 wherein said layer consists of material that loosens from said capsule body or dissolves at a temperature exceeding normal body temperature of the patient.

13. An endoscopy capsule as claimed in claim 10 wherein said cover is comprised of material that dissolves in a chemical reaction with a removal compound

14. An endoscopy capsule as claimed in claim 10 wherein said cover has a substantially uniform thickness of less than 0.1 mm.

15. An endoscopy capsule as claimed in claim 10 wherein said component is a video component having a field of view, and wherein said cover is optically transparent at least in a portion thereof in said field of view of said video component.

16. A method for reusing an ingestible endoscopy capsule, comprising the steps of:
providing an ingestible endoscopy capsule with an exchangeable cover at an exterior of the endoscopy capsule that protects the endoscopy capsule from the environment in a gastrointestinal tract of a patient during passage of the endoscopy capsule through the gastrointestinal tract; and
after passage of the endoscopy capsule through the gastrointestinal tract, removing said cover from said endoscopy capsule and replacing the removed cover with a replacement cover for subsequent reuse of the endoscopy capsule with the replacement cover.

17. A method as claimed in claim 16 comprising removing said cover by heating said cover to a temperature that exceeds a body temperature of the patient to loosen said cover from the endoscopy capsule or to dissolve said cover.

18. A method as claimed in claim 16 comprising removing said cover from said endoscopy capsule by applying a removal compound to said cover that dissolves said cover by a chemical reaction.

19. A method as claimed in claim 15 comprising employing a foil as said cover and removing said cover by tearing said foil away from said endoscopy capsule.

20. A method as claimed in claim 15 comprising applying said cover to said endoscopy capsule by an application procedure selected from the group consisting of spraying, lamination, and layering.
